(19) United States
(12) Patent Application Publication
(10) Pub. No.: US 2022/0104712 A1
(43) Pub. Date: Apr. 7, 2022

(54) AORTIC STENOSIS ECHOCARDIOGRAPHIC FOLLOW-UP EXPERT SYSTEM

(71) Applicants: KONINKLIJKE PHILIPS N.V., EINDHOVEN (NL); FUNDACION INSTITUTO DE ESTUDIOS DE CIENCIAS DE LA SALUD DE CASTILLA Y LEON (IECSCYL-IBSAL), SORIA (ES)

(72) Inventors: PEDRO LUIS SANCHEZ FERNANDEZ, SALAMANCA (ES); PEDRO IGNACIO DORADO DIAZ, SALAMANCA (ES); JESUS MANUEL SAMPEDRO GOMEZ, SALAMANCA (ES); VICTOR VICENTE PALACIOS, SALAMANCA (ES); JAVIER SANCHEZ GONZALEZ, MADRID (ES); LUIS CUEVAS SEMPERE, MADRID (ES)

(21) Appl. No.: 17/426,926

(22) PCT Filed: Jan. 30, 2020
(86) PCT No.: PCT/EP2020/052317

§ 371 Date: Jul. 29, 2021

Related U.S. Application Data
(60) Provisional application No. 62/798,504, filed on Jan. 30, 2019.

(51) Int. Cl. A61B 5/02; A61B 5/00; A61B 5/0285; G16H 50/50; G16H 50/30

(52) U.S. Cl. CPC A61B 5/02028; A61B 5/7267; A61B 5/742; G16H 50/30; A61B 5/0285; G16H 50/50; A61B 5/7475

(57) ABSTRACT

An imaging examination analysis method includes receiving a patient data set for a patient including measurements for a set of variables relating to a chronic medical condition, such as aortic stenosis (AS), obtained by an imaging examination of the patient. For one or more future time intervals, a likelihood is generated of a predetermined grade of the chronic medical condition (e.g. likelihood of severe AS grade) for the patient at the future time interval. The likelihood is generated using a classifier trained on training data sets for past patients. A follow-up imaging examination date recommendation is determined based on the likelihoods. An inconsistent measurement for the patient may be identified as a measurement whose value compared with a prior measurement for the same variable is consistent with an improvement in the chronic medical condition of the patient between the time of the prior measurement and a present time.

### FIELD

The following relates generally to the cardiology arts, echocardiography arts, cardiac imaging arts, patient scheduling arts, and related arts.

### BACKGROUND

Aortic Stenosis (AS) occurs when the aortic valve narrows. The grade of AS progresses over time from “mild”, to “moderate”, to “severe”, corresponding to increasing AS constriction. For AS graded as severe, intervention in the form of an aortic valve replacement is typically indicated. Transthoracic echocardiography is typically used to grade the AS, although another cardiac imaging modality such as MRI or CT may be used. Follow-up AS imaging examinations are scheduled at regular time intervals that typically depend on the AS grade (“mild”, “moderate”, or “severe”), with the time intervals decreasing as the AS grade worsens. A common follow-up interval schedule employed in Europe suggests performing a follow-up echocardiography examination every 6 months for severe AS, every 1 year for moderate AS, and every 1-3 years for mild AS. In contrast, somewhat different intervals are sometimes used in the United States, especially in initial AS stages, for example recommending a follow-up echocardiography every 6-12 months for severe AS, every 1-2 years for moderate AS, and every 3-5 years for mild AS.

The following discloses certain improvements.

### SUMMARY

In some non-limiting illustrative embodiments disclosed herein, a non-transitory storage medium stores instructions readable and executable by an electronic processor to perform an aortic stenosis imaging examination analysis method including: receiving a patient data set for the patient including measurements for a set of aortic stenosis variables obtained by an aortic stenosis imaging examination of the patient performed on an imaging examination date; for one or more future time intervals relative to the imaging examination date, generating a likelihood of a severe aortic stenosis grade for the patient at the future time interval; and determining a follow-up aortic stenosis imaging examination date recommendation based on the generated likelihoods. The likelihood(s) is/are generated by processing the patient data set using a classifier trained on training data sets for past patients including measurements for the set of aortic stenosis variables obtained by aortic stenosis imaging examinations of the respective past patients and labeled as to whether the respective past patients were diagnosed with severe aortic stenosis grades as of the future time interval relative to the respective imaging examination dates of the aortic stenosis imaging examinations of the respective past patients.

In some non-limiting illustrative embodiments disclosed herein, an aortic stenosis imaging examination device is disclosed, comprising an imaging device configured to perform an aortic stenosis imaging examination of a patient on an imaging examination date; an electronic processor, and a non-transitory storage medium storing instructions readable and executable by the electronic processor to perform an aortic stenosis imaging examination analysis method. The imaging device includes a display and one or more user input devices. The aortic stenosis imaging examination analysis method includes: providing an imaging device user interface via which one or more images of the aortic stenosis imaging examination are displayed on the display of the imaging device and via which measurements for a set of aortic stenosis variables are provided via inputs from the one or more user input devices of the imaging device; retrieving, from an electronic data storage, prior measurements for the set of aortic stenosis variables obtained by a prior aortic stenosis imaging examination of the patient performed prior to the imaging examination date; identifying an inconsistent measurement for the patient from the measurements for the set of aortic stenosis variables as a measurement whose value compared with the prior measurement for the same aortic stenosis variable in the retrieved prior measurements is consistent with a reduction of a constriction of the aortic valve of the patient between the time that the prior aortic stenosis imaging examination of the patient was performed and the imaging examination date; and displaying, on the imaging device user interface, a message indicating the inconsistent measurement.

In some non-limiting illustrative embodiments disclosed herein, an imaging examination analysis method is disclosed. A patient data set for a patient is received at an electronic processor. The patient data set includes measurements for a set of variables relating to a chronic medical condition obtained by an imaging examination of the patient performed on an imaging examination date. For one or more future time intervals relative to the imaging examination date a likelihood of a predetermined grade of the chronic medical condition for the patient at the future time interval is generated by processing operations performed by the electronic processor. The likelihood is generated by processing the patient data set using a classifier trained on training data sets for past patients including measurements for the set of variables relating to the chronic medical condition obtained by imaging examinations of the respective past patients and labeled as to whether the respective past patients were diagnosed with the predetermined grade as of the future time interval relative to the respective imaging examination dates of the imaging examinations of the respective past patients. A follow-up imaging examination date recommendation is determined for performing a follow-up imaging examination to assess the chronic medical condition for the patient based on the generated likelihoods.

One advantage resides in a population level reduction in the number of imaging examinations performed for monitoring AS.

Another advantage resides in anticipating follow-up imaging and associated treatments to patients with higher risk of accelerated deterioration.

Another advantage resides in reduced number of call back imaging examinations for monitoring AS.

Another advantage resides in providing a more efficient user interface for an imaging device used in monitoring AS.

Another advantage resides in providing more efficient scheduling of follow-up imaging examinations for monitoring AS.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 diagrammatically illustrates an aortic stenosis (AS) imaging examination system including an expert system providing measurement consistency checks and follow-up AS imaging examination scheduling.

FIG. 2 diagrammatically illustrates an AS monitoring method suitably performed by the AS monitoring system of FIG. 1.

FIG. 3 diagrammatically illustrates a feature selection procedure suitable for selecting features for input to the follow-up AS imaging examination scheduling component of the AS monitoring system of FIG. 1

FIG. 4 diagrammatically illustrates a suitable implementation of the follow-up AS imaging examination scheduling component of the AS monitoring system of FIG. 1.

FIG. 5 diagrammatically illustrates Receiver Operating Characteristic (ROCs) curves for an illustrative implementation of the follow-up AS imaging examination scheduling component of the AS monitoring system of FIG. 1.

### DETAILED DESCRIPTION

Aortic Stenosis (AS) occurs when the heart's aortic valve narrows, reducing blood flow from the heart into the aorta. AS is a prevalent valve disease and an important cause of morbidity and mortality. Monitoring by echocardiography is recommended for all patients with AS. Asymptomatic patients with AS should still undergo follow up since AS is an ongoing disease that progresses with time. Asymptomatic patients should undergo echocardiographic follow up at different intervals depending on the severity of the disease. AS is graded as mild, moderate or severe, for example using the illustrative guideline of Table 1 employing the aortic stenosis variables of aortic peak velocity, aortic mean gradient, and aortic valve area.

TABLE 1

| | Aortic Peak | Aortic Mean | Aortic Valve |
|---|---|---|---|
| AS grade | Velocity (m/s) | Gradient (mm Hg) | Area (cm²) |
| Mild | 2.5-2.9 | <20 | >1.5 |
| Moderate | 3-4 | 20-40 | 1-1.5 |
| Severe | >4 | >40 | <1 |

Every AS patient will eventually develop severe AS, if he or she does not decease in the interim due to comorbidities. Patients with severe AS are at increased risk of heart failure, syncope, angina or death and should ideally undergo aortic valve replacement before or shortly after they reach severe state. Another noteworthy situation is a case in which the patient develops left ventricular dysfunction. Left ventricular dysfunction is a critical situation for patients with AS and calls for further clinical evaluation. If AS is severe then this is a likely cause of the left ventricular dysfunction, and the patient typically should undergo valve replacement. If AS is not severe, then the left ventricular dysfunction may be due to some other disease or condition (e.g. coronary heart disease), and further clinical evaluation should be performed to identify or rule out such causes. In general, periodic monitoring with transthoracic echocardiography is recommended in asymptomatic patients with known AS at intervals depending on valve severity. Another cardiac imaging modality can alternatively be used, such as using a magnetic resonance imaging (MRI) scanner or a computed tomography (CT) scanner, but echocardiography tends to be a particularly cost-effective imaging modality. As noted previously, a common follow-up interval schedule employed in Europe suggests performing a follow-up echocardiography examination every 6 months for severe AS, every 1 year for moderate AS, and every 1-3 years for mild AS. In contrast, somewhat different intervals are sometimes used in the United States, especially in initial AS stages, for example recommending a follow-up echocardiography every 6-12 months for severe AS, every 1-2 years for moderate AS, and every 3-5 years for mild AS.

However, rigid follow-up imaging examination scheduling can be inefficient. It is disclosed herein that patients with stable AS can be characterized using Artificial Intelligence (AI) methods in order to schedule follow-up AS imaging examinations that are tailored to the AS progression of specific individual AS patients. By contrast, using existing rigid follow-up AS imaging examinations, stable patients sometimes receive unnecessary AS follow-up imaging, which introduces unnecessary costs to the medical care system and inconvenience and stress to AS patients. Avoiding only a portion of such redundant AS imaging examinations can lead to a significant reduction in hospital workload and societal cost.

Another source of unnecessary AS imaging is call-back AS imaging examinations. Embodiments disclosed herein perform consistency checks on the AS measurements to detect results that are inconsistent with the known progression in which the AS can only become worse over time (except in the case of an aortic valve replacement intervention, or possibly in the case of a patient who develops left ventricular dysfunction which calls for deep clinical evaluation as noted above). AS is a constriction of the aortic valve. Hence, put another way, the progression of AS is that the constriction of the aortic valve of the patient can only increase over time. As used herein, “increased constriction” refers to the blockage of blood flow becoming greater, that is, the increased constriction causes the lumen of the aortic valve through which the blood flows to become narrower. By contrast, a reduction of the constriction of the aortic valve of the patient would correspond to the blockage becoming smaller, that is, a reduced constriction would correspond to the lumen of the aortic valve through which the blood flows to becoming wider. Such a reduction of the constriction of the aortic valve is not expected in any AS patient since the AS can only worsen over time (again, except in the case of an aortic valve replacement intervention). (Note: A reduction of the constriction of the aortic valve is always expected in patients with AS, even in the case of left ventricular dysfunction and except where there has been an aortic valve intervention. But with left ventricular dysfunction, measurements of AS could vary and peak velocity could decrease as it depends on the stroke volume, etc.)

It is disclosed herein to perform consistency checks on AS measurements obtained by an aortic stenosis imaging examination on this basis. In this approach, an inconsistent measurement for the patient is identified as a measurement whose value compared with a prior measurement for the same aortic stenosis variable is consistent with a reduction of the constriction of the aortic valve of the patient between the time that the prior aortic stenosis imaging examination of the patient was performed and the current imaging examination date. Since such a reduction in the constriction is not physically realistic, an inconsistency is identified, and a message may be displayed on the imaging device display indicating the inconsistent measurement. By this approach, the imaging technician (or other qualified medical professional performing the AS imaging examination) is made aware of the inconsistency while the patient is still undergoing the AS imaging examination, so that the inconsistency can be investigated and potentially corrected (e.g., by repeating the imaging acquisition, or re-measuring the variable in the already-acquired cardiac image) so as to avoid a call-back examination. By way of non-limiting illustrative example, an aortic velocity or gradient variable whose current measurement is lower than a prior measurement for the same aortic velocity or gradient variable is an inconsistency, since lower velocity corresponds to less constriction which is not physically realistic. As another non-limiting illustrative example, an aortic valve area variable whose current measurement is higher than a prior measurement for the same aortic valve area is an inconsistency, since again the indicated increase in lumen cross-sectional area over time is not a physically realistic AS progression.

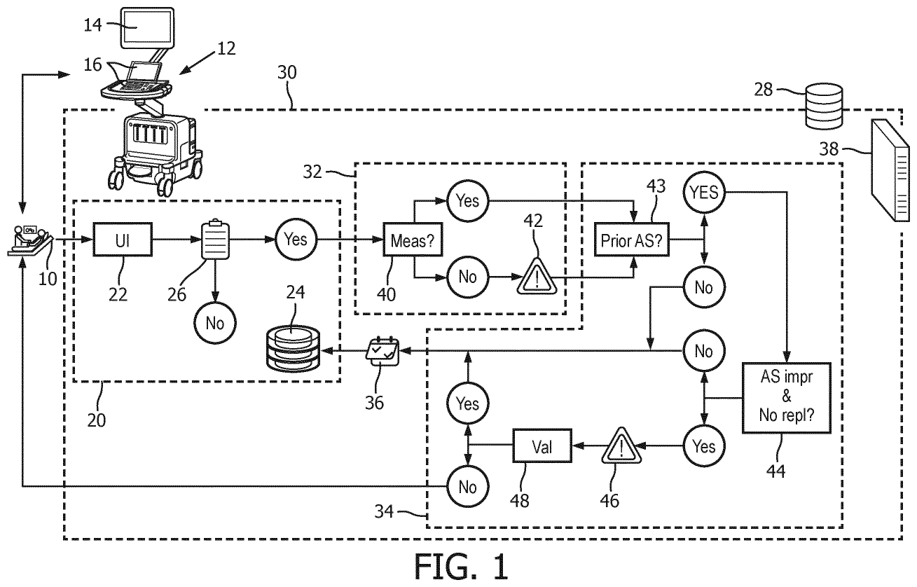

With reference to FIG. 1, an AS imaging examination system is diagrammatically shown, which includes an expert system providing a follow-up AS imaging examination recommendation and also includes measurement consistency and/or missing measurement detection. The illustrative system includes a cardiac imaging device 10, which in the illustrative example is a cardiac ultrasound imaging system (i.e. echocardiograph) 12 programmed to perform cardiac imaging. Alternatively, the imaging device 10 could be an MRI scanner, a CT scanner, or any other imaging modality capable of acquiring cardiac images from which measurements can be obtained for a set of AS variables for characterizing aortic stenosis (e.g., aortic velocity variables, aortic valve area variables, where “area” refers to the cross-sectional area of the lumen of the aortic valve through which blood flow passes, calcium measurement of the valve, and/or so forth. As shown by way of the illustrative echocardiograph device 12, the cardiac imaging device 10 typically includes a display 14 for presenting cardiac images and/or for displaying AS measurements and so forth, and one or more user input devices 16 (e.g. keyboard, trackball/mouse/trackpad or other pointing device, a touch screen, dictation microphone, and/or so forth). The AS imaging examination system further includes AS imaging workflow components 20 by which a cardiologist, imaging technician, or other qualified medical personnel interpret and report echocardiograms. The illustrative workflow components 20 include a user interface 22 via which one or more images of the aortic stenosis imaging examination (where the images are acquired by the imaging device 10) are displayed on the display 14 and via which the measurements for a set of aortic stenosis variables are provided via inputs from one or more user input devices 16. The illustrative workflow components 20 further include a non-transitory data storage medium 24 (e.g., a magnetic disk or other magnetic storage medium, a solid state drive or other electronic storage medium, and/or so forth) that stores the image(s) of the AS imaging examination and various associated data (e.g. the measurements for the set of aortic stenosis variables, additional demographic data on the patient, patient management data such as a unique patient identifier, and/or so forth). The illustrative workflow components 20 further include an implementation of AS guidelines 26 for assessing whether the patient has AS and, if so, grading the AS, based on the measurements for the set of AS variables obtained from the AS imaging examination of the patient and possibly also based on other patient data such as demographic data. The illustrative workflow components 20 may be implemented using any suitable cardiology and/or medical imaging informatics system comprising software stored on a non-transitory storage medium 28 and running on (that is, the instructions are read and executed by) an electronic processor 30. For example, the illustrative workflow components 20 may be implemented by the IntelliSpace Cardiovascular (ISCV) management solution (software available from Koninklijke Philips N. V.) stored on the non-transitory storage medium 28 and running on the electronic processor 30. As disclosed herein, the cardiology informatics components are extended by providing a combination of further components 32, 34, 36 that implement aspects of aortic stenosis imaging examination analysis. These further components include one or more of a measurements completeness component 32, a measurements consistency check component 34, and/or a follow-up AS imaging examination scheduling component 36.

It will be appreciated that the non-transitory storage medium 28 and the electronic processor 30 may be variously implemented and/or variously distributed. For example, the electronic processor 30 may include one or more of: an electronic processor of the imaging device 10 or an associated imaging controller (e.g. a desktop computer or the like connected to control the imaging device); an illustrative network-based server computer 38; an ad hoc network of computers implementing a cloud computing resource (not shown); various combinations thereof; and/or so forth. Similarly, the non-transitory storage medium 28 may include one or more of: a RAID or other network-based magnetic storage; a solid state drive (SSD) or other electronic data storage; an optical disk or other optical data storage; various combinations thereof; and/or so forth. It will be appreciated that execution of the various functions and corresponding distribution of execution of the software implementing those functions may be distributed over various electronic processors (hence “an electronic processor” as used herein is to be understood as encompassing both embodiments including a single electronic processor and embodiments including a combination of two or more electronic processors); and likewise, storage of the software for implementing those functions may be distributed over one, two, or more physical non-transitory storage media (hence “a non-transitory storage medium” as used herein is to be understood as encompassing both embodiments including a single non-transitory storage medium and embodiments including a combination of two or more non-transitory storage media). For example, in some embodiments processing related to presenting the user interface 22 and to the consistency and completeness checks 32, 34 are performed by the electronic processor of the illustrative echocardiograph 12, while the more computationally complex processing of the AS imaging examination scheduling component 36 is performed by the network-based server computer 38 (or by a cloud computing resource or other electronic processor(s) with large processing capacity).

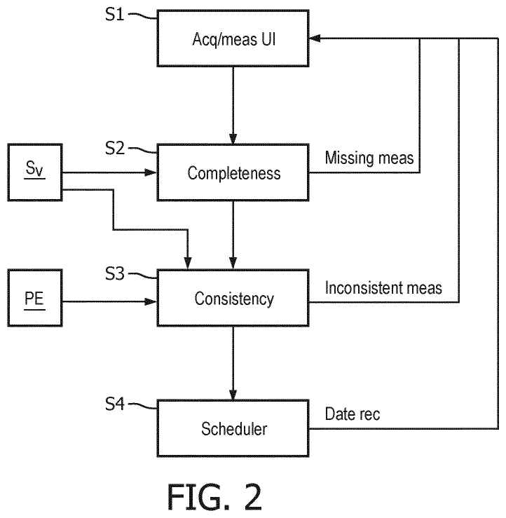

With continuing reference to FIG. 1 and with brief reference to FIG. 2, an illustrative AS imaging examination analysis workflow is shown at a high level. In an operation S1, the image(s) of the AS imaging examination of the patient are acquired, and the clinician operates the user interface 22 to visually review cardiac image(s) displayed on the 14 and to provide the measurements for the set of aortic stenosis variables via inputs from one or more user input devices 16. The providing of the measurements should be broadly construed as encompassing a range of ways by which the measurements may be provided, such as: typing in measurement values; manipulating cursors superimposed on displayed cardiac images to graphically delineate a measurement such as an aortic valve diameter (where an additional or different parameter, namely the aortic valve area, may be computed as πr² where r is one-half of the graphically measured aortic valve diameter i.e., r is the aortic valve radius); selecting via a user dialog to accept a measurement that is calculated from the AS image(s) by the electronic processor; receiving a measurement that is automatically calculated from the AS image(s) by the electronic processor without such a user accept operation; and/or so forth. Those measurements could also be automatically obtained by Artificial Intelligence image analysis device.

After the measurements are fully entered, in an operation S2 performed by the completeness check component 32, the received AS measurements are compared against a set of AS variables Sᵥ to identify any missing measurements, and if a missing measurement is thereby identified then a message indicating the missing AS variable is displayed on the user interface 22. Preferably, the clinician will then perform the missing measurement using the already-acquired cardiac images, or if necessary will acquire additional cardiac images providing the data for performing the missing measurement(s). The set of AS variables Sᵥ preferably includes all AS variables that are considered important for performing AS grading. Put another way, the set of AS variables Sᵥ preferably includes every AS variable that, if not measured in the current AS imaging examination, might to trigger a call-back examination to obtain that missing measurement.

In an operation S3 performed by the consistency check component 34, the received AS measurements are compared against a prior AS imaging examination PE, if such a prior examination PE is available. The consistency check S3 determines whether the progression of any AS variable(s) between the prior examination PE and the current examination is not physically realistic. In the case of AS grading, it is expected that (in the absence of an aortic valve replacement or diagnosed left ventricular dysfunction) the constriction of the aortic valve of the patient can only stay the same or become worse over time (that is, more constricted, i.e. the constriction increases whereby the area of the valve lumen through which blood flows is decreased). Hence, any measurement whose value compared with the prior measurement for the same aortic stenosis variable in the retrieved prior measurements is consistent with a reduction of the constriction of the aortic valve between the time that the prior aortic stenosis imaging examination PE of the patient was performed and the imaging examination date of the current AS imaging examination is not a physically realistic progression of the AS chronic condition. As specific examples: any velocity measurement that is lower in the current measurement is not physically realistic (this is because a more constricted aortic valve results in the blood flow velocity increasing due to its being more compressed laterally by the narrowed aortic valve lumen). Similarly, any aortic gradient measurement that is lower in the current measurement is not physically realistic (the rationale being analogous to that for velocity variables). On the other hand, any valve diameter or valve area measurement that is larger in the current measurement is not physically realistic (this is a direct measurement that the valve is less constricted in the current measurement, which is not physically realistic). If an inconsistent measurement is thereby identified then a message indicating the inconsistent measurement is displayed on the user interface 22. Preferably, the clinician will review the situation, to determine whether the current measurement (or, perhaps, the prior measurement) is in error. If the current measurement is suspected to be in error, then it is preferably re-measured (including reacquiring the underlying image(s) if deemed appropriate). The consistency check S3 is preferably performed for any AS measurement in the set of AS variables Sᵥ for which a consistency check can be defined (e.g., for which the current measurement is expected to increase, or decrease, over time based on the expected progression of the chronic condition). An analogous consistency test can be performed for the AS grade assigned by the clinician (e.g., as represented by an AS grade finding code (FC) in some systems). If the clinician assigns the patient with a better AS grade in the current AS imaging examination compared with the prior examination PE then this can be flagged as inconsistent (again, assuming that no aortic valve replacement was performed between the prior and current imaging examinations—this can be checked by accessing the Electronic Record of the patient in the Cardiovascular Information System or other relevant patient database). By way of illustration, if for the current imaging examination the clinician assigns the patient's AS a grade of “mild” but the prior examination includes a FC indicating the patient AS was graded as “moderate”, this is an inconsistency that should be flagged.

It is additionally/alternatively contemplated for the consistency check S3 performed by the consistency check component 34 to employ other types of consistency checks. For example, a measurement can be checked against a credible range for the values of the measurement, and any incredible measurement thereby flagged as an inconsistent measurement. For example, aortic area variables are expected to have positive values, and there may also be a realistic upper range value set for each variable which, if exceeded, is flagged as an inconsistent measurement. These types of checks also enable unrealistic AS measurements to be identified so as to encourage performing a re-measurement while the patient is undergoing the AS imaging examination, so as to reduce or eliminate call-back examinations.

With continuing reference to FIGS. 1 and 2, an operation S4 performed by the AS imaging examination scheduling component 36, a follow-up AS imaging examination date recommendation is generated, and displayed on the user interface 22. Operation of some illustrative embodiments of the AS imaging examination scheduling component 36 will be described in more detail with reference to FIGS. 1 and 3-5; however, in general a machine learning component trained on past patients who have undergone AS imaging examinations is employed to generate estimates for one or more future time intervals of the likelihood(s) that the patient will have a severe AS grade as of those respective future time intervals (measured relative to the date of the current AS imaging examination). Hence, if the machine learning component estimates that the patient has a low likelihood (e.g. below some threshold) of progressing to severe AS at a one year interval, but a high likelihood (e.g., above the threshold) of progressing to severe AS at a two year interval, then the AS imaging examination scheduling component 36 may suitably recommend scheduling the follow-up AS imaging examination at two years from the current date of the present AS imaging examination.

In some embodiments, the user interface 22 for embodiments of the disclosed AS imaging examination analysis expert system is provided at the echocardiograph device 12, MRI scanner, CT scanner, or other cardiac imaging device 10 used to perform the AS imaging examination (for example, provided using the display 14 and user input device(s) 16 of the illustrative echocardiograph 12). In this way, the clinician receives immediate feedback on any missing or inconsistent AS measurements in the operations S2, S3, so as to allow for immediate correction without the need to schedule a call-back examination, and the next AS imaging examination may be scheduled in the operation S4 immediately upon completion of the current AS imaging examination, in accordance with the follow-up AS examination schedule recommendation provided by the expert system 36 on an individualized basis. To facilitate this latter feature, in some embodiments the artificial intelligence (AI) component 36 that provides the schedule recommendation receives only AS imaging measurements as inputs, which are readily available at the imaging device 10. (Such exclusivity of the patient data for use in the schedule recommendation is merely an optional feature; in other contemplated embodiments, additional inputs such as patient demographic data may be received by the AI component 36 as well, and used by the AI component 36 in making the schedule recommendation).

It may also be noted that the AS imaging examination analysis may incorporate any single one of these operations S2, S3, S4, or may incorporate any two of these operations S2, S3, S4, or may incorporate all three of these operations S2, S3, S4 (as in the illustrative example of FIGS. 1 and 2).

With returning reference to FIG. 1, an illustrative embodiment of the completeness check component 32 is described. A typical echocardiography reporting system includes different data elements. Quantitative measurements (e.g., the AS measurements) are derived from the cardiac image(s). This may be done manually, or with computer-aided measurement support (possibly including artificial intelligence measurement determination components). In the IntelliSpace Cardiovascular (ISCV) system, image processing tooling such as TomTec Arena or QLab may be used for this purpose. Measurements may also be acquired autonomously. The clinician may also input structured diagnostic codes, i.e. finding codes (FCs), which are structured codes associated with particular diagnoses. Internally, a report can then be represented as a series of FCs. From a series of FCs, a natural language and human readable report is generated by mapping each FC onto its natural language representation. Additionally or alternatively, the clinician may input report information manually using a free text format. As diagrammatically shown in FIG. 1, the AS imaging workflow components 20 may assess whether a patient has AS by checking cardiac measurements and/or FCs pertaining to AS against the AS guidelines 26. If either data source contains evidence that the patient has AS, then the processing operations S2, S3, S4 of FIG. 2 are performed by respective components 32, 34, 36 as diagrammatically shown in FIG. 1. In some embodiments, the determination that the patient has AS may be stored in an Electronic Health Record, Electronic Medical Record, Cardiovascular Information System, or other central database for future access, so the AS determination can be made by accessing this prior determination.

The illustrative completeness check component 32 checks in the echo reporting environment 20 to determine if all input value fields have been entered. If one or more measurements are missing (block 40), this can be highlighted by providing a suitable message 42 in the echo reporting environment and/or in a dedicated panel, e.g., as error messages or by relaying to the user that an imputed value is used until further notice. In some embodiments, feedback is provided to the user as to what is the impact of missing data on the accuracy of the recommendation, e.g., in the form of an error interval. These messages 42 encourage the cardiologist to specify all relevant input values (e.g., all those in the set of AS variables Sᵥ, see FIG. 2).

The illustrative consistency check component 34 checks at operation 43 whether a prior AS imaging examination for the patient is available (for example, retrievable from the non-transitory data storage medium 24). If no such prior examination is available, then flow passes to the AS imaging examination scheduling component 36. On the other hand, if a prior AS imaging examination is available in the non-transitory data storage medium 24 (e.g., corresponding to the prior examination PE of FIG. 2), then at operation 44 each measurement whose consistency is to be checked is examined to determine whether the current measurement indicates the patient's AS has improved compared with the prior examination. If so, this is an inconsistent measurement, and is flagged as previously described for the operation S3. If at operation 44 any measurement (or other checked information, such as the FC indicating the AS grade assigned by the clinician) indicates that the AS of the patient has improved between the prior imaging examination and the current imaging examination, then a message 46, 48 is displayed on the user interface 22 indicating the inconsistent measurement (or FC).

Some illustrative embodiments of the AS imaging examination scheduling component 36 are next described.

The AS imaging examination scheduling component 36 suitably employs a deep neural network, support vector machine, or other artificial intelligence (AI) model that takes a patient data set including (by way of illustrative example): AS measurements (e.g. echocardiograph measurements when using the illustrative echocardiograph 12), or a history thereof; qualitative assessments, e.g., in the form of finding codes such as an AS grade FC, or a history thereof; patient demographics; and/or so forth. As noted previously, in some embodiments only AS measurements available at the imaging device 10 are the inputs. As output the AI model predicts if a patient's AS condition will progress to severe within a pre-determined time interval (e.g., 1 year, 2 years, 3 years). Multiple models can be created for different intervals. These models can be combined into one decision tree to suggest a follow-up interval:
If patient AS condition is predicted to progress to severe within next 1 year, return <1 yr
Else if patient AS condition is predicted to progress to severe within next 2 years, return <2 yr
Else if patient AS condition is predicted to progress to severe within next 3 years, return <3 yr
Else return >=3 yr

Various AI models and training techniques can be employed. Some suitable AI models include Random Forests, XGBoost, convolutional Neural Networks (“deep learning”) and logistic regression. In one embodiment, the AI model is developed once and is used as is for all deployment sites. In another embodiment, the AI model is localized (i.e. trained) based on the database of the hospital where the model will be deployed. In this embodiment, a pre-selected set of input parameters can be used, or a dedicated input selection step is implemented (e.g., principal component analysis).

While the illustrative examples employ the illustrative echocardiograph 12 as the AS imaging modality, if some other imaging modality is used then the model is suitably trained on AS measurements by that modality and hence ingests such data. Hence, in another embodiment employing the computed tomography (CT) imaging modality, the AI model takes measurements on cardiac CT, or a history thereof. In another embodiment employing magnetic resonance (MR) imaging, the AI model takes measurement on cardiac MR, or a history thereof. It is also contemplated to employ an AI model that uses measurements acquired using two or more different imaging modalities, if such extensive AS imaging examination measurements are routinely available for patients at a given medical institution. The patient data set input to the AI model may include other patient data such as current medications (e.g., may be relevant if the patient is taking a medication that is known to have an adverse side effect pertaining to AS), most recent selected lab values, or a history thereof, additional lifestyle information from wearables, co-morbidities in the form of International Classification of Diseases (ICD) codes, and/or so forth.

In some embodiments, the AS imaging examination scheduling component 36 converts the follow-up AS imaging examination date recommendation into a finding code (FC) and/or a natural language recommendation. The recommendation can be entered automatically into the report before finalization, or can be suggested to the user for insertion. In another contemplated output embodiment, the recommendation is stored in a structured database and follow-up is monitored by an agent. In yet another contemplated embodiment, the AS imaging examination scheduling component 36 is operatively connected with the patient scheduling system of the relevant imaging department (e.g. the cardiology department or so forth) so that the patient can schedule the follow-up AS imaging examination immediately upon completion of the current AS imaging examination.

In another contemplated variant, the AS imaging examination scheduling component 36 interacts with the workflow components 20 to detect a manually entered follow-up AS imaging examination date against the AS imaging examination date recommendations output by the AI model. To do so, it deploys natural language processing techniques to (1) detect sentences that make a follow-up recommendation and (2) extract the recommended interval (1 year, 2 years, etc.). This functionality can be implemented using natural language processing (NLP) processing using, for instance, regular expression methods, to detect keywords and linguistic variations. If the actual scheduled follow-up date differs significantly from the AI recommendation, this may be recorded in a database and/or used to automatically update training of the AI model (in a dynamic adaptation variation of the AI model; in some embodiments, the update training will wait until the follow-up examination is actually performed so as to have the ground truth information as to whether the patient had progressed to the severe AS grade at the time of the follow-up imaging examination). Additionally or alternatively, a dialogue can be initiated with the cardiologist, checking whether the cardiologist prefers to retain the original recommendation or wishes to replace it with the recommendation suggested by the AI.

The AI model for the AS imaging examination scheduling component 36 suitably operates as follows. For one or more future time intervals relative to the imaging examination date (of the current AS imaging examination), a likelihood is generated of a severe aortic stenosis grade for the patient at the future time interval. The likelihood may be a true probability (normalizing to unity), or more generally may be an unnormalized indication of how likely it is that the patient will be graded as having severe AS at the future time interval. The likelihood is suitably generated by processing the input patient data set (including at least measurements for a set of AS variables obtained by the AS imaging examination of the patient performed on the imaging examination date) using a classifier (i.e., the AI model). The classifier is trained on training data sets for past patients including measurements for the set of AS variables obtained by AS imaging examinations of the respective past patients and labeled as to whether the respective past patients were diagnosed with severe AS grades as of the future time interval relative to the respective imaging examination dates of the AS imaging examinations of the respective past patients. A follow-up aortic stenosis imaging examination date recommendation is determined based on the generated likelihoods.

In some embodiments, the generating of the likelihood is sequentially performed for one or more successively larger future time intervals until the generated likelihood for a last-tested future time interval exceeds a threshold, and the follow-up aortic stenosis imaging examination date recommendation is determined as the last-tested future time interval.

In some embodiments, the set of aortic stenosis variables includes at least one aortic velocity variable, such as a peak aortic jet velocity, a mean aortic velocity, an aortic velocity time integral, and/or so forth.

In some embodiments, the patient data set includes at least five variables of the group consisting of peak aortic jet velocity, mean aortic velocity, aortic velocity time integral, patient age, left ventricle mass, slope of deceleration of mitral E wave, ejection fraction, stroke volume, mean left ventricular outflow track velocity, and telediastolic volume.

In some embodiments, the patient data set comprises variables including peak aortic jet velocity, mean aortic velocity, aortic velocity time integral, patient age, left ventricle mass, slope of deceleration of mitral E wave, ejection fraction, stroke volume, mean left ventricular outflow track velocity, and telediastolic volume.

These are merely some non-limiting illustrative examples of suitable contents of the patient data set.

As noted in the example above, the follow-up aortic stenosis imaging examination date recommendation is determined based on the generated likelihoods, e.g. as the last-tested future time interval in the above more particular example. The recommendation may be variously determined and presented. For example, the recommendation may be to perform a follow-up examination in one year; or, it may be presented as an actual date, for example if the current AS imaging examination is performed on Jan. 30, 2021 then the recommendation may be to schedule the follow-up AS imaging examination for Jan. 30, 2022. However, as that is a Sunday, it is alternatively contemplated for the recommendation may be to schedule the follow-up AS imaging examination for Monday, Jan. 31, 2022 or some other nearby weekday. Said another way, a recommendation to perform the follow-up in one year does not necessarily mean exactly one year from the current date, but rather may be adjusted by a reasonable amount to fall on a weekday, to avoid other schedule conflicts, and/or so forth. In other embodiments, e.g. when the follow-up recommendation is for a one-year follow-up, this may not designate the precise day the follow-up examination will be scheduled.

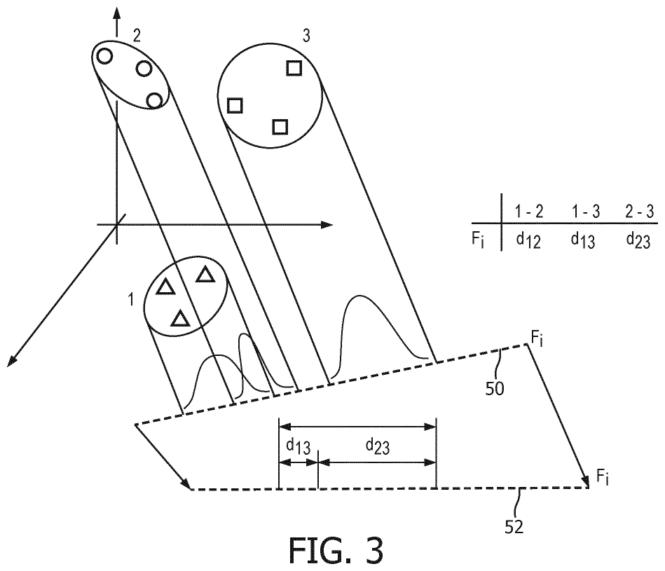

With reference now to FIGS. 3-5, a worked-out non-limiting illustrative example of a suitable AI model for use in the AS imaging examination scheduling component 36 is described. The echocardiogram data used in this example included a total amount of 96,146 echocardiograms, of which 5,595 correspond to AS patients who have had at least two echocardiograms performed (so that a prior examination is available). The data was organized and structured, and a dataset was built that consisted in 346 features that derived from the measurements of the echocardiographic image and basic clinical data (age, gender, weight and height). The initial and final stage of the severity of the AS (according to the European and American guidelines) and the residence times for each patient in each of them.

Prior to training the actual model, a feature selection operation was performed. Traditional Machine Learning (ML) feature selection techniques such as “Permutation Importance” usually give a general response. Classic univariate statistical analysis (Chi-Square or ANOVA test) that can lead to Type I-II errors. To avoid this limitation the illustrative worked-out AI example employed a combination of Multivariate Analysis Of Variance (MANOVA) and Biplot methods, permitting simultaneous hyperspatial representations of the different groups to be compared (severe/non-severe AS); the different variables under analysis. This method includes t-test based on Wilk's Lambda distribution, a probability distribution used in multivariate hypothesis testing. It is a multivariate generalization of the univariate F-distribution similar to Student's t-distribution.

With reference to FIG. 3, a hyperspatial representation 50 of both groups and variables are obtained, and each group (severe/non-severe AS) is algebraically projected onto each variable. The standardized subtraction of the resulting group projections onto one variable represents the resulting discriminant score for this variable. By extension, a ranked list 52 was retrieved of this discriminant scores by analyzed feature. From the feature selection analysis, the set of AS variables for ingestion by the AI model were selected. The set of AS variables for the worked-out example included: peak aortic jet velocity, mean aortic velocity, aortic velocity time integral, patient age, left ventricle mass, slope of deceleration of mitral E wave, ejection fraction, stroke volume, mean left ventricular outflow track velocity, and telediastolic volume. Again, this is merely a non-limiting illustrative example, and additional, fewer, and/or other variables may be included in the set of AS variables.

In summary, 32 variables were selected through which the AI model was trained. It was found for this worked-out example that a minimum of 10 variables were needed to obtain a good outcome. Assurance of quality control of these 10 measurements is advantageous in order to ensure good predictions. Such quality control can be provided, for example, by the completeness and consistency check components 32, 34 of illustrative FIG. 1.

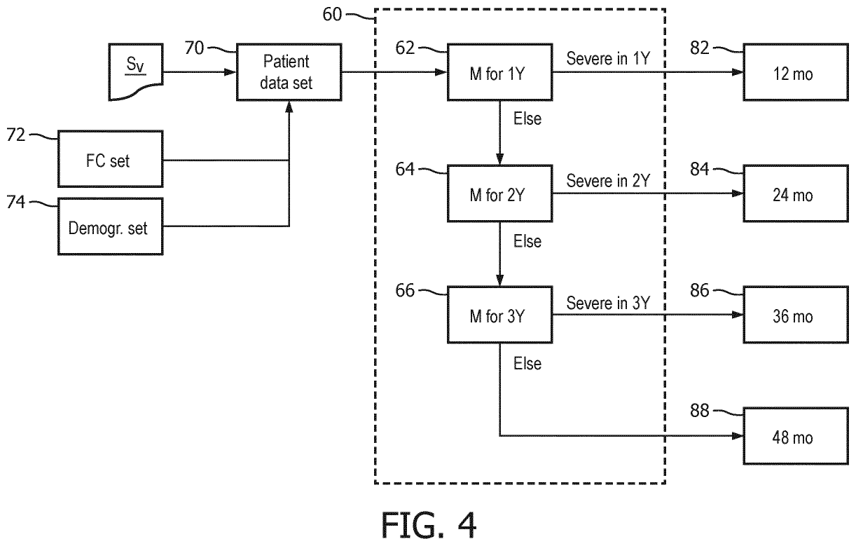

With reference now to FIG. 4, a classifier 60 trained on the above-selected set of features is diagrammatically illustrated. The illustrative classification model enchained three different classifiers 62, 64, 66. The inputs to the worked-out classifier 60 included a patient data set 70 consisting of the variables selected by the feature selection algorithm discussed above; more generally, as illustrated in FIG. 4, the patient data set 70 may include measurements 72 from the echocardiogram, finding codes (FCs) 72, demographic data 74, and/or so forth. Each classifier 62, 64, 66 was trained on the above-described training data to predict whether a patient will develop or not severe AS within: one year (classifier 62), two years (classifier 64), or three years (classifier 66). To produce the worked-out example, several machine learning algorithms were tested (logistic regression, decision trees, random forests, support vector machines, among others) with the aim of maximizing the recall of the prediction (number of true positive predictions divided by number of real positive values). The goal was to detect as many patients who will not progress to be graded at severe AS within the designated one, two, or three year future time interval as possible without missing the ones who will worsen to the severe grade in this future time interval. The classifying algorithm with the best performance, and used in the final models, was XGBoost. Moreover, as the dataset obtained was imbalanced, due to having more non-severe AS cases than severe ones, a ML technique called Balanced Bagging Classifier was applied. The XGBoost and Imblearn open source libraries of these algorithms implemented in Python were used.

With continuing reference to FIG. 4, it is seen that the enchained arrangement of the three classifiers 62, 64, 66 to form the overall classifier 60 implements the approach of sequentially performing for one or more successively larger future time intervals (one year by classifier 62, then two year by classifier 64, then three year by classifier 66) until the generated likelihood for a last-tested future time interval exceeds a threshold. In this approach, the one year classifier 62 is first applied. If the likelihood generated by the classifier 62 exceeds the threshold, then this is the last-tested future time interval and the follow-up aortic stenosis imaging examination date recommendation is determined as a one year (i.e. 12 month) time interval 82. Otherwise (i.e. else), the two year classifier 64 is next applied. If the likelihood generated by the classifier 64 exceeds the threshold, then this is the last-tested future time interval and the follow-up aortic stenosis imaging examination date recommendation is determined as a two year (i.e. 24 month) time interval 84. Otherwise (i.e. else), the three year classifier 66 is next applied. If the likelihood generated by the classifier 66 exceeds the threshold, then the follow-up aortic stenosis imaging examination date recommendation is determined as a three year time (i.e. 36 month) interval 86. Otherwise (i.e. else), the follow-up aortic stenosis imaging examination date recommendation is determined as a four year (i.e. 48 month) time interval 88. It will be appreciated that this is merely an illustrative arrangement, and other classifier topologies and parameters may be used. For example, other follow-up time intervals can be used instead of one-, two-, three-, and four-years, by training the constituent classifiers on those different future time intervals. Moreover, the enchained approach can included more or fewer than three enchained classifiers so as to provide coarser or finer time resolution, respectively, for the follow-up recommendation. Still further, other classifier topologies besides the illustrative enchained topology are contemplated.

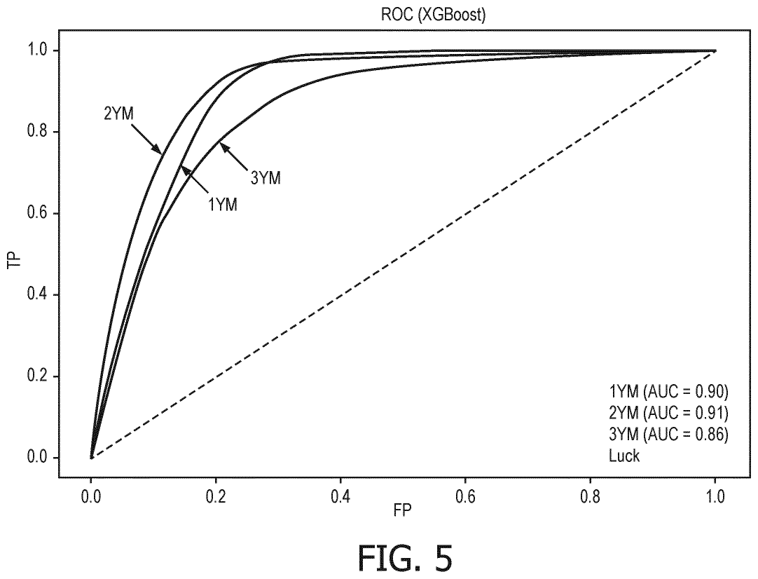

With reference to FIG. 5, the likelihood threshold used in the classifier 60 was tuned to obtain the desired performance by generating Receiver Operating Characteristic curves (ROCs) as shown in FIG. 5 to select the threshold. The models were evaluated by 50 randomized repetitions using two thirds of the dataset as training set and the remaining third as a test set. Areas under ROC curves of 0.90, 0.91 and 0.86 (FIG. 5) and accuracies of 0.78, 0.82 and 0.77 were obtained by one year model 62, two year model 64, and three year model 66, respectively. In addition to these outcomes, the model had a 75% rate of false positive results. Nevertheless, a rate of 88% of true positive results was achieved, which leads to the conclusion that the worked out AI model is robust. The sequential algorithm (i.e. the enchained classifiers 62, 64, 66 of FIG. 4) accurately detects the non-severe/severe AS transitions in exchange of a too early prediction.

To test the performance of the classifier 60 as trained in the worked-out example, the optimal visit (ideal situation) was compared with the scheduled visits provided by the trained classifier 60. It was found that 67.43% of the cases were correctly assigned. Two different errors were analyzed: minor and major errors. Minor errors are those whose result is prior to the ideal appointment, and are tabulated in Table 2. Major errors are those whose visit is scheduled after the ideal one, and are tabulated in Table 3. Major errors are of more clinical significance, as they amount to a delay in diagnosing when the patient progresses to severe AS. Major errors represent less of the 3% of the cases.

TABLE 2

| Error type | Years before | % |
|---|---|---|
| | 1 | 14.29% |
| Minor | 2 | 5.8% |
| | 3 | 10.19% |

TABLE 3

| Error | Years after | % |
|---|---|---|
| Major | 1 | 1.7% |
| | 2 | 0.6% |
| | 3 | 0.0% |

A significant utility of the disclosed personalized follow-up scheduling lies in the fact that the current fixed American and European guidelines for follow-up AS imaging examinations are conservative in that they tend to schedule more frequent follow-up examinations than necessary, especially for patients whose AS is stable. The disclosed AS imaging examination scheduling component 36 enables the follow-up time interval to be optimized, especially for patients with AS graded as mild, while the classifiers also detect patients that are likely to develop severe AS more rapidly. In other words, the AI model is as conservative as the clinical guidelines with the patients that will develop severe AS rapidly, but is more efficient with the remainder of the cases. Table 4 provides estimates made herein of the number of echocardiography studies that could be saved by leveraging the disclosed AS imaging examination scheduling component 36.

TABLE 4

| Model | Mild | Moderate | Total |
|---|---|---|---|
| EU guideline | 92.69% | 12.34% | 41.5% |
| US guideline | 28.46% | 12.34% | 18.19% |

The illustrative embodiments are directed to aortic stenosis imaging examination analysis. However, it will be appreciated that the disclosed measurements completeness component 32, a measurements consistency check component 34, and/or a follow-up imaging examination scheduling component 36, can be readily employed in conjunction with any type of chronic medical condition that whose progression over time is monitored by medical imaging examinations scheduled at time intervals. One such application was mentioned above, namely detection of the patient developing left ventricular dysfunction. In this case the disclosed system may be configured to identify this issue as different variables in the system are related to left ventricular function, such as ejection fraction, stroke volume, telediastolic volume, and/or so forth. Some further non-limiting examples of chronic medical conditions that are monitored by medical imaging and for which the disclosed approaches can be used to provide follow-up imaging examination schedule recommendations are provided in the following.

Aortic regurgitation (AR) is a chronic medical condition that is also commonly monitored by echocardiography in order to quantity AR and measurements of left ventricle function and dimensions. Present clinical guidelines recommend serial follow-up examinations for asymptomatic patients. These guidelines again exhibit inconsistences between American practice (every 1 year for severe, every 1-2 year for moderate, every 3-5 years for mild) and European practice (mild/moderate echocardiography every 2 years, severe every year). As with AS, other imaging modalities such as CT and/or MRI may be used in place of the usual echocardiography in the follow-up imaging examinations.

Mitral regurgitation is a chronic medical condition that is also commonly monitored by echocardiography. Asymptomatic patients with severe mitral regurgitation and normal left ventricular ejection fraction (LVEF) should be monitored using echocardiography. Guidelines also recommend serial follow-up examinations for asymptomatic patients with inconsistences between American (every 6 months—1 year for severe, every 1-2 year for moderate, every 3-5 years for mild) and European guidelines (mild/moderate echocardiography every 2 years, severe every year). Mitral stenosis (MS) is yet another illustrative non-limiting chronic medical condition that is also commonly monitored by echocardiography. Guidelines also recommend serial echocardiographic follow-up examinations for asymptomatic patients with inconsistences between American (every 1 year for severe, every 1-2 year for moderate, every 3-5 years for mild) and European guidelines (moderate echocardiography every 2-3 years, severe every year).

Embodiments of the disclosed expert follow-up scheduling recommendation systems are readily employed for such conditions as the above-mentioned AR, mitral regurgitation, or MS, and/or for other chronic medical conditions such as pulmonary valve stenosis, pulmonary valve regurgitation, tricuspid stenosis, tricuspid regurgitation, or so forth, for biological valve assessment of various valves (aortic, mitral, tricuspid, pulmonary), mechanical valve assessment of various valves (aortic, mitral, tricuspid, pulmonary), and/or so forth. To implement the follow-up scheduling recommendation expert system for a given chronic medical condition, the follow-up imaging examination scheduling component 36 more generally receives, at the electronic processor 30, a patient data set 70 for the patient including measurements for a set of variables Sᵥ relating to the chronic medical condition (AS in the illustrative examples) obtained by an imaging examination of the patient performed on an imaging examination date. For one or more future time intervals relative to the imaging examination date, a likelihood of a predetermined grade of the chronic medical condition for the patient at the future time interval is generated by processing operations performed by the electronic processor 30. The likelihood is generated by processing the patient data set 70 using a classifier 60 trained on training data sets for past patients including measurements for the set of variables Sᵥ relating to the chronic medical condition obtained by imaging examinations of the respective past patients and labeled as to whether the respective past patients were diagnosed with the predetermined grade as of the future time interval relative to the respective imaging examination dates of the imaging examinations of the respective past patients. A follow-up imaging examination date recommendation is determined for performing a follow-up imaging examination to assess the chronic medical condition for the patient based on the generated likelihoods.

Likewise, the measurements consistency check component 34 is generally applicable to any chronic medical condition whose progression is expected to become worse over time (absent some intervention analogous to the aortic valve replacement for treating severe AS). As some other non-limiting illustrative examples, some chronic medical conditions commonly monitored by CT and/or MRI include valve heart disease monitoring for various cardiac valves (e.g. aortic, mitral, tricuspid, pulmonary), where CT can for example quantify calcium in the valves (which is a metric of the valve calcification), and where MRI can for example quantify the left ventricular volumes and fibrosis. CT and/or MRI can also provide additional or alternative information on the dimensions and geometry of the aortic root and ascending, descending, and abdominal aorta. Thus the monitored chronic medical condition could be (as further non-limiting examples) an aneurysm of the aorta or of another major blood vessel, whose progression is expected to become worse over time and where surgical or percutaneous treatment is also recommended. In such cases, the consistency check entails retrieving, from an electronic data storage 24 to the electronic processor 30, prior measurements for the set of variables Sᵥ relating to the chronic medical condition obtained by a prior imaging examination of the patient performed prior to the imaging examination date. By processing operations performed by the electronic processor 30, an inconsistent measurement for the patient is identified from the measurements for the set of variables Sᵥ relating to the chronic medical condition as a measurement whose value compared with the prior measurement for the same variable in the retrieved prior measurements is consistent with an improvement in the chronic medical condition of the patient between the time that the prior aortic stenosis imaging examination of the patient was performed and the imaging examination date. (Again, the rationale for this identification is that the chronic medical condition is of a type which is expected to only become worse over time). A message 46, 48 indicating the inconsistent measurement is displayed on a display.

The invention has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory storage medium storing instructions readable and executable by an electronic processor to perform an aortic stenosis imaging examination analysis method including:
receiving a patient data set for the patient including measurements for a set of aortic stenosis variables (Sᵥ) obtained by an aortic stenosis imaging examination of the patient performed on an imaging examination date;
for one or more future time intervals relative to the imaging examination date, generating a likelihood of a severe aortic stenosis grade for the patient at the future time interval wherein the likelihood is generated by processing the patient data set using a classifier trained on training data sets for past patients including measurements for the set of aortic stenosis variables obtained by aortic stenosis imaging examinations of the respective past patients and labeled as to whether the respective past patients were diagnosed with severe aortic stenosis grades as of the future time interval relative to the respective imaging examination dates of the aortic stenosis imaging examinations of the respective past patients; and
determining a follow-up aortic stenosis imaging examination date recommendation based on the generated likelihoods.

2. The non-transitory storage medium of claim 1 wherein the set of aortic stenosis variables (Sᵥ) includes at least one aortic velocity variable.

3. The non-transitory storage medium of claim 1 wherein the patient data set includes at least five variables of the group consisting of peak aortic jet velocity, mean aortic velocity, aortic velocity time integral, patient age, left ventricle mass, slope of deceleration of mitral E wave, ejection fraction, stroke volume, mean left ventricular outflow track velocity, and telediastolic volume.

4. The non-transitory storage medium of claim 1 wherein the patient data set comprises variables including peak aortic jet velocity, mean aortic velocity, aortic velocity time integral, patient age, left ventricle mass, slope of deceleration of mitral E wave, ejection fraction, stroke volume, mean left ventricular outflow track velocity, and telediastolic volume.

5. The non-transitory storage medium of claim 1 wherein the patient data set for the patient includes only the measurements for the set of aortic stenosis variables (Sᵥ) obtained by the aortic stenosis imaging examination of the patient performed on the imaging examination date.

6. The non-transitory storage medium of claim 1 wherein the aortic stenosis imaging examination analysis method further includes:
displaying the follow-up aortic stenosis imaging examination date recommendation on a display operatively connected with the electronic processor.

7. The non-transitory storage medium of claim 1 wherein:
the generating is sequentially performed for one or more successively larger future time intervals until the generated likelihood for a last-tested future time interval exceeds a threshold; and the follow-up aortic stenosis imaging examination date recommendation is determined as the last-tested future time interval.

8. The non-transitory storage medium of claim 1 wherein the aortic stenosis imaging examination analysis further includes:
providing a user interface via which one or more images of the aortic stenosis imaging examination are displayed on a display and via which the measurements for the set of aortic stenosis variables (Sᵥ) are provided via inputs from one or more user input devices.

9. The non-transitory storage medium of claim 8 wherein the aortic stenosis imaging examination analysis further includes:
retrieving, from an electronic data storage, prior measurements for the set of aortic stenosis variables (Sᵥ) obtained by a prior aortic stenosis imaging examination (PE) of the patient performed prior to the imaging examination date;
in the patient data set, identifying an inconsistent measurement for the patient from the measurements for the set of aortic stenosis variables as a measurement whose value compared with the prior measurement for the same aortic stenosis variable in the retrieved prior measurements is consistent with a reduction of a constriction of the aortic valve of the patient between the time that the prior aortic stenosis imaging examination of the patient was performed and the imaging examination date; and
displaying, on a display via the user interface, a message indicating the inconsistent measurement.

10. The non-transitory storage medium of claim 9 wherein the identifying includes identifying at least one of:
an aortic velocity or gradient variable whose value is lower than the prior measurement for the same aortic velocity or gradient variable in the retrieved prior measurements; or
an aortic valve area variable whose value is higher than the prior measurement for the same aortic valve area variable in the retrieved prior measurements.

11. The non-transitory storage medium of claim 8 wherein the receiving of the patient data set includes:
identifying a missing aortic stenosis variable of the set of aortic stenosis variables (Sᵥ) for which the received patient data set does not include a measurement; and
displaying, on a display via the user interface, a message indicating the missing aortic stenosis variable.

12. An aortic stenosis imaging examination device comprising:
an imaging device configured to perform an aortic stenosis imaging examination of a patient on an imaging examination date, the imaging device including a display and one or more user input devices;
an electronic processor; and
a non-transitory storage medium storing instructions readable and executable by the electronic processor to perform an aortic stenosis imaging examination analysis method including:
providing an imaging device user interface via which one or more images of the aortic stenosis imaging examination are displayed on the display of the imaging device and via which measurements for a set of aortic stenosis variables (Sᵥ) are provided via inputs from the one or more user input devices of the imaging device;
retrieving, from an electronic data storage, prior measurements for the set of aortic stenosis variables obtained by a prior aortic stenosis imaging examination of the patient performed prior to the imaging examination date;
identifying an inconsistent measurement for the patient from the measurements for the set of aortic stenosis variables as a measurement whose value compared with the prior measurement for the same aortic stenosis variable in the retrieved prior measurements is consistent with a reduction of a constriction of the aortic valve of the patient between the time that the prior aortic stenosis imaging examination of the patient was performed and the imaging examination date; and
displaying, on the imaging device user interface, a message indicating the inconsistent measurement.

13. The aortic stenosis imaging examination device of claim 12 wherein the identified inconsistent measurement includes an aortic velocity or gradient variable whose value is lower than the prior measurement for the same aortic velocity or gradient variable in the retrieved prior measurements.

14. The aortic stenosis imaging examination device of claim 12 wherein the identified inconsistent measurement includes an aortic valve area variable whose value is higher than the prior measurement for the same aortic valve area variable in the retrieved prior measurements.

15. The aortic stenosis imaging examination device of claim 12 wherein the aortic stenosis imaging examination analysis method further includes:
for one or more future time intervals relative to the imaging examination date, generating a likelihood of a severe aortic stenosis grade for the patient at the future time interval wherein the likelihood is generated by processing a patient data set including at least the measurements for the set of aortic stenosis variables (Sᵥ) using a classifier trained on training data sets for past patients including measurements for the set of aortic stenosis variables obtained by aortic stenosis imaging examinations of the respective past patients and labeled as to whether the respective past patients were diagnosed with severe aortic stenosis grades as of the future time interval relative to the respective imaging examination dates of the aortic stenosis imaging examinations of the respective past patients;
determining a follow-up aortic stenosis imaging examination date recommendation based on the generated likelihoods; and
displaying, on the imaging device user interface, the follow-up aortic stenosis imaging examination date recommendation.

16. The aortic stenosis imaging examination device of claim 15 wherein:
the generating is sequentially performed for one or more successively larger future time intervals until the generated likelihood for a last-tested future time interval exceeds a threshold; and the follow-up aortic stenosis imaging examination date recommendation is determined as the last-tested future time interval.

17. The aortic stenosis imaging examination device of claim 12 wherein the imaging device is a cardiac ultrasound imaging system, a magnetic resonance imaging scanner, or a computed tomography scanner.

18. The aortic stenosis imaging examination device of claim 17 wherein the imaging device is a cardiac ultrasound imaging system.

19. An imaging examination analysis method including:
receiving, at an electronic processor, a patient data set for a patient including measurements for a set of variables (Sᵥ) relating to a chronic medical condition obtained by an imaging examination of the patient performed on an imaging examination date;
for one or more future time intervals relative to the imaging examination date, generating, by processing operations performed by the electronic processor, a likelihood of a predetermined grade of the chronic medical condition for the patient at the future time interval wherein the likelihood is generated by processing the patient data set using a classifier trained on training data sets for past patients including measurements for the set of variables relating to the chronic medical condition obtained by imaging examinations of the respective past patients and labeled as to whether the respective past patients were diagnosed with the predetermined grade as of the future time interval relative to the respective imaging examination dates of the imaging examinations of the respective past patients; and
determining a follow-up imaging examination date recommendation for performing a follow-up imaging examination to assess the chronic medical condition for the patient based on the generated likelihoods.

20. The imaging examination analysis method of claim 19 further comprising:
retrieving, from an electronic data storage to the electronic processor, prior measurements for the set of variables (Sᵥ) relating to the chronic medical condition obtained by a prior imaging examination of the patient performed prior to the imaging examination date;
identifying, by processing operations performed by the electronic processor, an inconsistent measurement for the patient from the measurements for the set of variables relating to the chronic medical condition as a measurement whose value compared with the prior measurement for the same variable in the retrieved prior measurements is consistent with an improvement in the chronic medical condition of the patient between the time that the prior aortic stenosis imaging examination of the patient was performed and the imaging examination date; and
displaying, on a display, a message indicating the inconsistent measurement.

21. The imaging examination analysis method of claim 19 wherein the chronic medical condition is aortic stenosis, aortic regurgitation, mitral regurgitation, mitral stenosis, pulmonary valve stenosis, pulmonary valve regurgitation, tricuspid stenosis, or tricuspid regurgitation, biological valve assessments, mechanical valve assessments, left ventricular dysfunction, dilatation or aneurysm of great vessels including aorta.
